(19) United States
(12) Patent Application Publication
(10) Pub. No.: US 2025/0017779 A1
(43) Pub. Date: Jan. 16, 2025

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/766,913

(22) Filed: Jul. 9, 2024

(30) Foreign Application Priority Data
Jul. 11, 2023 (DE) 10 2023 118 293.5

(51) Int. Cl. A61F 9/008

(52) U.S. Cl. CPC A61F 9/00802; A61F 2009/00872

(57) ABSTRACT

The invention relates to a method for providing control data for an ophthalmological laser (12) of a treatment apparatus (10) for treating a cornea (16) of an eye. Hereto, a control device (18) of the treatment apparatus (10) may ascertain (S10) eye parameters from predetermined examination data; determine (S12) a respective diameter for respective optical zones (OZ) depending on the ascertained eye parameters, wherein a diameter for treating the cornea (16) is selected from the ascertained diameters of the optical zones (OZ); and provide (S14) control data, which includes the selected diameter of the optical zone (OZ).

### FIELD

The invention relates to a method for providing control data for an ophthalmological laser of a treatment apparatus for treating a cornea of an eye. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device may for example be formed such that laser pulses effect a photodisruption and/or ablation in a focus area situated within the organic tissue, to remove a tissue, in particular a tissue lenticule, from the cornea.

In such a treatment, an optical zone and a transition zone adjoining thereto are usually set as a treatment area. Herein, the optical zone provides the planned diameter for the treatment, in which the optically effective zone is to be performed for changing the optical characteristics of the cornea. The transition zone adjoining thereto is intended to provide a gentle transition from the separated tissue of the optical zone to the remaining corneal tissue. Usually, the choice of the diameter of the optical zone is freely selectable in treatment apparatuses and is set according to empirical values. However, they do not have to match optimum diameters for optical zones since multiple factors are in particular to be taken into account in setting an optimized diameter for the optical zone.

### SUMMARY

Therefore, it is the object of the invention to simplify and/or to improve planning for treating a cornea of an eye, in particular setting a diameter of an optical zone.

This object is solved by the independent claims. Advantageous embodiments of the invention are disclosed in the dependent claims, the following description as well as the figures.

The invention is based on the idea that diameters for optical zones are automatically provided, which are optimized for the treatment, depending on the treatment to be performed, which may be ascertained from predetermined eye parameters. For example, at least one diameter may be ascertained for the optical zone and/or diameter ranges may be provided, that is a range of diameters, which are between two diameter values, and which are optimized for the planned treatment.

An aspect of the invention relates to a method for providing control data for an ophthalmological laser of a treatment apparatus for treating a cornea of an eye, wherein the method comprises the following steps performed by a control device. Therein, an appliance or an appliance component, in particular a computer or a processor, may be understood by a control device, which may automatically perform the following steps according to specification of predetermined parameters and/or in which individual steps may be performed according to user inputs: ascertaining eye parameters from predetermined examination data; ascertaining a respective diameter for respective optical zones depending on the ascertained eye parameters by the control device, wherein a diameter for treatment of the cornea is selected from the ascertained diameters of the optical zones; and providing control data, which includes the selected diameter of the optical zone.

In other words, predetermined examination data may first be provided to the control device, for example by transferring the examination data from an examination device to the control device via a wireless and/or wired connection. Alternatively or additionally, the examination data may also be manually input into the control device via a user interface. The control device may determine eye parameters from it, such as, for example, visual disorder data, in particular myopia values, hyperopia values, astigmatism values and/or values for a spherical aberration. Furthermore, values for a pupil size, in particular in scotopic conditions or a maximized pupil, may, for example, also be provided in the eye parameters. For example, information about pretreatments of the eye and/or a currently planned treatment may also be provided in the eye parameters.

From these eye parameters, the control device may determine at least one diameter for an optical zone, in particular in automated manner, which is optimized based on the previously ascertained eye parameters. For example, this may be performed by look-up tables and/or preset calculation formulas and/or decision trees. For example, multiple diameters may be provided, wherein each diameter defines a further optical zone, wherein these optical zones may all be optimized for the ascertained eye parameters. This means that multiple diameters may be present, which are possible for a treatment, wherein they may then be provided to a user for selection. Then, a user may select one of the provided diameters for the treatment of the cornea for example via a user interface. For example, if only one diameter is ascertained for the optical zone by the control device, it may be selected by the control device, in particular in automated manner.

Then, the selected diameter of the optical zone may be provided to the treatment apparatus in the form of control data, which effects a treatment of the cornea of the eye with the selected diameter of the optical zone upon execution of the control data.

For example, diameters for the optical zone may be provided by the control device, which provide a change of a corneal curvature by reducing a regression and/or a regrowth of an epithelial layer of the cornea and which are smaller than the diameter of the cornea.

By the invention, the advantage arises that the planning of a corneal treatment is facilitated for a user because the optimized diameters are automatically determined depending on the eye parameters and thus further considerations and/or calculations are not required for the user. Furthermore, the treatment results may thus be improved since the optimized diameters may always be used for the optical zones.

The invention also includes embodiments, by which additional advantages arise.

In an embodiment, the eye parameters include at least one pupil diameter, in particular a pupil diameter of a scotopic pupil, wherein diameters are provided for the optical zone, which are larger than the pupil diameter. This means that the diameters of the provided optical zones at least cover the area of the pupil, in particular of a pupil with maximized diameter. Herein, the diameters of the optical zones may for example be limited downwards by the ascertained pupil diameter and upwards by the diameter of the cornea, and alternatively or additionally, the diameters of the optical zones may be limited towards the top depending on further parameters, which may be ascertained from the eye parameters. By this embodiment, the advantage arises that in particular in poor lighting conditions, such as for example in darkness, impairments do not occur after the treatment.

In a further embodiment, a correction type is determined from the eye parameters, in particular myopia correction or hyperopia correction, wherein, for correction data in which a main portion of an ablation is effected in a peripheral position of the cornea, larger diameters of the optical zone are provided than for correction types in which the main portion of the ablation is effected in a central position of the cornea. A central position of the cornea may, for example, be set by a pupil central point and/or a corneal vertex and a peripheral position radially spaced from this center. For example, if a hyperopia correction is planned, a main portion of a focus of the ablation may be effected in a peripheral position of the cornea since a lenticule in the form of a diverging lens is removed from the cornea to compensate for the hyperopic portion. In such a hyperopia correction, therein, larger diameters may be provided for the optical zone than, for example, for a myopia correction, in which more tissue is centrally removed than peripherally. Hereby, it is meant by a main portion of the ablation, that at least 60%, for example, more than 80%, of the tissue to be removed is in peripheral positions. In particular, diameters may be provided for the optical zone in transepithelial ablations, in which a treatment of a stroma is ensured. By this embodiment, the advantage arises that the diameters for the optical zone may be optimized depending on the planned correction.

In a further embodiment, three diameter ranges are preset, wherein a first diameter range with diameters larger than a first diameter value, a second diameter range with diameters in the range from the first diameter value down to a second diameter value, wherein the first diameter value is larger than the second diameter value, and a third diameter range with diameters smaller than the second diameter value are provided, wherein one of the three diameter ranges with the associated diameters is set depending on the eye parameters. This means that the diameters present in the provided diameter range may subsequently be offered for selection. In other words, the control device may provide diameters, which are within one of the three preset diameter ranges. Therein, a first or upper diameter range includes diameters above a first diameter value and a third or lower diameter range includes diameters below a second diameter value. In a second or middle diameter range, diameters are provided, which are within the range of the first and second diameter values. Thus, that diameter range with the diameters situated therein may be set by the control device, which best matches the present eye parameters.

For the diameter ranges, the first diameter value may be 7 mm and the second diameter value may be 6.5 mm. This means that the first diameter range includes diameters larger than 7 mm, the second diameter range includes diameters from 6.5 mm to 7 mm and the third diameter range includes diameters smaller than 6.5 mm. These diameter values represent statistically ascertained limit values, which have turned out to be particularly advantageous for the treatment of respective conditions described below.

In a further embodiment the first diameter range is provided, if one or more of the following conditions apply:
an astigmatism is above a preset astigmatism value, in particular greater than or equal to 2.5 diopters;
a second treatment for a myopia correction is present, after a myopia correction has been performed as a first treatment;
a second treatment for a hyperopia correction is present, after a hyperopia correction has been performed as a first treatment;
a myopia with a spherical aberration above 0.25 diopters is present;
a hyperopia with a spherical aberration below 0 diopters is present.

The second diameter range may be provided if one or more of the following conditions apply:
a first treatment for a myopia correction is performed;
a first treatment for a hyperopia correction is performed;
a second treatment for a myopia correction is present, after a hyperopia correction has been performed as a first treatment;
a myopia with a spherical aberration below 0.25 diopters is present;
a hyperopia with a spherical aberration in a range from 0 diopters to 0.25 diopters is present.

The third diameter range may be provided if one or more of the following conditions apply:
a second treatment for a hyperopia correction is performed after a myopia correction has been performed as a first treatment;
a hyperopia with a spherical aberration above 0.25 diopters is present.

Accordingly, the control device may set which one of the three diameter ranges is determined, in the form of a look-up table or a decision tree, to provide the diameters situated therein for the optical zone for selection.

In a further embodiment, the following calculations are performed for ascertaining the diameters of the optical zone:

$$\begin{matrix}O {Z}_{1} = 7.6 + 0.15 * min ( Sph ; Sph + Cyl ; 0 ) ; \\ O {Z}_{2} = 7.8 - 0.3 * max ( Sph ; Sph + Cyl ; 0 ) ; \\ O {Z}_{3} = 7.5 - 0.25 * abs ( Cyl ) ;\end{matrix}$$

wherein OZ₁, OZ₂ and OZ₃ are respective diameter values for the optical zone, Sph is a spherical refraction value and Cyl is a cylindrical refraction value, wherein a minimum value from OZ₁, OZ₂ and OZ₃ or a maximum value from OZ₁, OZ₂ and OZ₃ or an average value from OZ₁, OZ₂ and OZ₃ is selected as the diameter. The decision if the minimum value, maximum value or average value is selected, may for example be performed depending on the planned treatment, in particular if corneal volume is to be saved, a treatment result is to be maximized or a compromise between the above mentioned is to be provided.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is formed to perform the steps of at least one embodiment of one or both of the previously described methods. Thereto, the control device may comprise a computing unit for electronic data processing such as for example a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser may be formed to at least partially separate a predefined corneal volume with predefined interfaces of a human or animal eye by optical breakthrough, in particular at least partially separate it by photodisruption and/or to ablate corneal layers by (photo) ablation and/or to effect a laser-induced refractive index change in the cornea and/or the eye lens and/or to increase a crosslinking of the cornea.

In a further embodiment of the treatment apparatus according to the invention, the laser may be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

In addition to the diameter of the optical zone, the control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment;

FIG. 2 depicts a schematic method diagram for providing control data according to an exemplary embodiment.

### DETAILED DESCRIPTION

In the figures, identical or functionally identical elements are provided with the same reference characters.

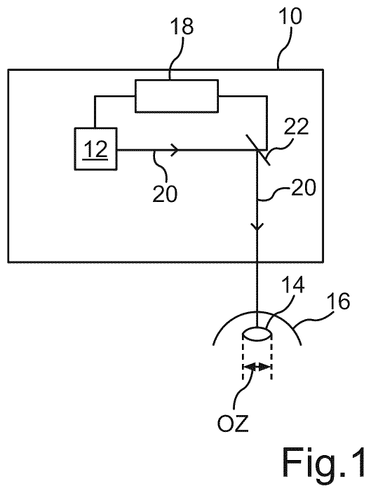

FIG. 1 shows a schematic representation of a treatment apparatus 10 with an ophthalmological laser 12 for removing a volume body 14 from a human or animal cornea 16 by photodisruption and/or ablation. For example, the volume body 14 may represent a lenticule, which may be separated from the cornea 16 by the eye surgical laser 12 for correcting a visual disorder. A correction profile or a geometry of the volume body 14 to be removed, which in particular includes a diameter of an optical zone OZ in which the optically effective change for visual disorder correction occurs, may be provided or ascertained by a control device 18, in particular in the form of control data, such that the laser 12 emits laser pulses in a pattern predefined by the control data into the cornea 16 of the eye to remove the volume body 14. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as, for example, a rotation scanner, to remove the volume body 14. The beam deflection device 22 may also be controlled by the control device 18 to remove the volume body 14.

In particular, the illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

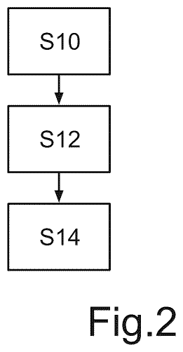

In removing the volume body 14 from the cornea 16, in particular, the choice of the diameter of the optical zone OZ plays a great role for the treatment success. Therein, the size of the optical zone OZ is firstly freely selectable, wherein it has been determined that according to eye parameters, for example, during a type of the refraction correction and/or pretreatments of the cornea 16, optimized diameters may be selected for the optical zone OZ, but which are not immediately apparent. In order to assist a user in setting the diameter of the optical zone and thereby to improve the treatment, the method shown in FIG. 2 may be performed by the control device 18 and/or by a control device external to the treatment apparatus 10, which for example belongs to a planning device.

FIG. 2 shows a schematic method diagram for providing control data for an ophthalmological laser 12 of a treatment apparatus 10 for treating a cornea 16 of an eye.

In a step S10, eye parameters may be determined from predetermined examination data, wherein the examination data may be provided from previously performed diagnostic measurements. Herein, the eye parameters relate to characteristics of the eye to be treated and/or of the cornea 16 such as for example a pupil diameter, in particular in a scotopic pupil, visual disorder data of the eye, in particular myopia, hyperopia and astigmatism values with respective spherical aberrations, and/or which treatment is to be performed and which treatments have already been performed.

In a step S12, at least one diameter of the optical zone OZ may be ascertained by the control device, which is optimized for the ascertained eye parameters. In particular, it may be provided that multiple diameters are determined, wherein a respective diameter provides a further optical zone OZ for the treatment.

In order to determine the optimized diameter of the optical zone OZ, the control device may consider various eye parameters. For example, a minimum diameter of the optical zone OZ may be set based on the pupil diameter, in particular of the scotopic pupil. As a further criterion, the correction type, which is to be performed for the cornea 16, may, for example, be ascertained, wherein in particular for a hyperopia correction, in which a main portion of the ablation is effected in a peripheral position of the cornea, a larger diameter is provided for the optical zone OZ than for a myopia correction.

In particular, at least three diameter ranges with diameters smaller than 6.5 mm, with diameters larger than 7 mm and diameters between 6.5 mm and 7 mm may be recorded in the control device 18. Depending on the present eye parameters, it may then be decided by the control device 18, which diameter range is offered for selection, wherein a diameter range with diameter values larger than 7 mm is for example provided on one or more of the following conditions:
an astigmatism is above a preset astigmatism value, in particular greater than or equal to 2.5 diopters;
a second treatment for a myopia correction is present, after a myopia correction has been performed as a first treatment;
a second treatment for a hyperopia correction is present, after a hyperopia correction has been performed as a first treatment;
a myopia with a spherical aberration above 0.25 diopters is present;
a hyperopia with a spherical aberration below 0 diopters is present

If one or more of the following conditions are present, a diameter range with values between 6.5 mm and 7 mm may be provided by the control device 18:
a first treatment for a myopia correction is performed;
a first treatment for a hyperopia correction is performed;
a second treatment for a myopia correction is present, after a hyperopia correction has been performed as a first treatment;
a myopia with a spherical aberration below 0.25 diopters is present;
a hyperopia with a spherical aberration in a range from 0 diopters to 0.25 diopters is present

The diameter range with diameters below 6.5 mm may be provided for selection if one of the following conditions is present:
a second treatment for a hyperopia correction is performed after a myopia correction has been performed as a first treatment;
a hyperopia with a spherical aberration above 0.25 diopters is present

Alternatively or additionally, the following calculation formulas may also be recorded in the control device 18, by which the optimized diameter for a respective treatment may be ascertained:

$$\begin{matrix}O {Z}_{1} = 7.6 + 0.15 * min ( Sph ; Sph + Cyl ; 0 ) ; \\ O {Z}_{2} = 7.8 - 0.3 * max ( Sph ; Sph + Cyl ; 0 ) ; \\ O {Z}_{3} = 7.5 - 0.25 * abs ( Cyl ) ;\end{matrix}$$

wherein OZ₁, OZ₂ and OZ₃ are respective diameter values for the optical zone, Sph is a spherical refraction value and Cyl is a cylindrical refraction value. Herein, the numerical values may be derived from statistics, which have been applied to preceding patient data, in particular from fit values. After OZ₁, OZ₂ and OZ₃ have been determined, a minimum value, a maximum value or an average value from these values may, for example, be determined to provide the diameter for the optical zone OZ.

In particular, a minimum value may be used if volume saving is desired, a maximum value in case of a maximized correction result and the average value in case of a compromise between volume saving and maximization of the treatment result. Thus, a nomogram may in particular be calculated, in which an optimized diameter for the optical zone OZ may be read from a desired refractive power correction. Thus, the following pairs of values of refractive power correction to diameter of the optical zone may for example be obtained:

(−18 D; 5.5 mm), (−15 D; 5.9 mm), (−12 D; 6.2 mm), (−9 D; 6.6 mm), (−6 D; 7.0 mm), (−3 D; 7.4 mm), (2 D; 7.5 mm), (4 D; 6.9 mm), (6 D; 6.3 mm), (8 D; 5.7 mm).

In particular, in transepithelial ablations, the meridional refractive power may also be related to the uncertainty of the epithelial thickness to ensure that a relevant part of the correction is generated in the stroma. This means that larger diameters may be planned by the control device 18 hereto, such as for example the following pairs of values:

(−15 D; 6.3 mm), (−10 D; 6.5 mm), (−4 D; 6.8 mm), (−2 D; 7.0 mm), (−1 D; 7.5 mm), (1 D; 8.0 mm), (2 D; 7.5 mm), (3 D; 7.2 mm), (4 D; 7.0 mm).

After the diameters for the optical zone OZ have been ascertained, they may be provided to a user, who may select a treatment diameter for treating the cornea 16.

Finally, control data may be provided in a step S14, by which the laser 12 and/or the beam deflection device 22 may be controlled to remove the volume body 14 from the cornea 16, wherein the volume body 14 includes the selected diameter of the optical zone OZ.

Overall, the examples show how a diameter for an optical zone OZ may be ascertained in improved manner by the invention.

1. A method for providing control data for an ophthalmological laser of a treatment apparatus for treating a cornea of an eye, wherein the method comprises the following steps performed by a control device:
ascertaining eye parameters from predetermined examination data;
ascertaining respective diameters for respective optical zones depending on the ascertained eye parameters by the control device, wherein a treatment diameter for treating the cornea is selected from the ascertained diameters of the optical zones; and
providing control data, which includes the selected diameter of the optical zone (OZ).

2. The method according to claim 1, wherein the eye parameters include at least one pupil diameter, wherein diameters are provided for the optical zone, which are larger than the at least one pupil diameter.

3. The method according to claim 1, wherein a correction type is determined from the eye parameters, wherein for correction types in which a main portion of an ablation is effected in a peripheral position of the cornea, larger diameters for the respective optical zones are ascertained than for correction types in which the main portion of the ablation is effected in a central position of the cornea.

4. The method according to claim 1, wherein three diameter ranges are preset, wherein a first diameter range with diameters larger than a first diameter value, a second diameter range with diameters in the range from the first diameter value down to a second diameter value, wherein the first diameter value is larger than the second diameter value, and a third diameter range with diameters smaller than the second diameter value are provided, wherein, depending on the eye parameters, one of the three diameter ranges is set by the control device.

5. The method according to claim 4, wherein the first diameter value is 7 mm and the second diameter value is 6.5 mm.

6. The method according to claim 4, wherein the first diameter range is provided if one or more of the following conditions apply:
an astigmatism above a preset astigmatism value;
a second treatment for a myopia correction, after a myopia correction has been performed as a first treatment;
a second treatment for a hyperopia correction, after a hyperopia correction has been performed as a first treatment;
a myopia with a spherical aberration above 0.25 diopters; and
a hyperopia with a spherical aberration below 0 diopters;
wherein the second diameter range is provided if one or more of the following conditions apply:
a first treatment for a myopia correction;
a first treatment for a hyperopia correction;
a second treatment for a myopia correction, after a hyperopia correction has been performed as a first treatment; and
a myopia with a spherical aberration below 0.25 diopters;
a hyperopia with a spherical aberration in a range from 0 diopters to 0.25 diopters;
wherein the third diameter range is provided if one or more of the following conditions apply:
a second treatment for a hyperopia correction, after a myopia correction has been performed as a first treatment; and
a hyperopia with a spherical aberration above 0.25 diopters.

7. The method according to claim 1, wherein the following calculations are performed for ascertaining the respective diameters of the optical zone (OZ):
$$\begin{matrix}O {Z}_{1} = 7.6 + 0.15 * min ( Sph ; Sph + Cyl ; 0 ) ; \\ O {Z}_{2} = 7.8 - 0.3 * max ( Sph ; Sph + Cyl ; 0 ) ; and \\ O {Z}_{3} = 7.5 - 0.25 * abs ( Cyl ) ;\end{matrix}$$
wherein OZ₁, OZ₂, and OZ₃ are respective diameter values for the optical zone, Sph is a spherical refraction value and Cyl is a cylindrical refraction value, wherein
a minimum value from OZ₁, OZ₂ and OZ₃ or
a maximum value from OZ₁, OZ₂ and OZ₃ or
an average value from OZ₁, OZ₂ and OZ₃ is selected as the treatment diameter.

8. The method according to claim 1, further including the following steps:
transferring the provided control data to a respective ophthalmological laser of the treatment apparatus; and
controlling the laser with the control data.

9. A control device, which is configured to perform the method according to claim 1.

10. A treatment apparatus with at least one ophthalmological laser for the separation of a corneal volume of a human or animal eye by optical breakdown and at least one control device according to claim 9.

11. (canceled)

12. A computer-readable medium for storing a computer program thereon, the computer program comprising commands which cause a treatment apparatus to execute the method according to claim 1.
